(19) United States
(12) Patent Application Publication
(10) Pub. No.: US 2026/0335083 A1
(43) Pub. Date: Oct. 29, 2026

(54) DEVICES AND METHODS FOR ACTIVE SIZING IN CARDIAC ANNULOPLASTY

(71) Applicants: Ramesh S. Veeragandham, Dublin, CA (US); Albert K. Chin, Palo Alto, CA (US)

(72) Inventors: Ramesh S. Veeragandham, Dublin, CA (US); Albert K. Chin, Palo Alto, CA (US)

(21) Appl. No.: 19/661,661

(22) Filed: Apr. 28, 2026

Related U.S. Application Data
(60) Provisional application No. 63/795,636, filed on Apr. 28, 2025.

(51) Int. Cl. A61F 2/24

(52) U.S. Cl. CPC A61F 2/2496; A61F 2/2466

(57) ABSTRACT

An active annuloplasty sizing device comprising an outer tube having a proximal end and a distal end, and an inner member slidably engaged with the outer tube and capable of extending from the proximal end of the outer tube. A sizer ring is coupled to a distal end of the outer tube, the sizer ring configured to be positioned at a valve annulus and to receive one or more suture ties for temporary attachment to the valve annulus. The sizer ring comprises an actuator tube and an actuator extending through the actuator tube and coupled to the inner member. Retraction of the inner member relative to the outer tube causes the actuator to decrease a circumference of the sizer ring. Methods of sizing a cardiac valve annulus and kits for cardiac annuloplasty sizing are also provided.

### INCORPORATION BY REFERENCE TO ANY PRIORITY APPLICATIONS

This application claims benefit of priority to U.S. Provisional Application No. 63/795,636, filed Apr. 28, 2025, which is expressly incorporated by reference in its entirety for all purposes.

### BACKGROUND

### Field

The field of the present disclosure corresponds to cardiac valve repair devices, more particularly active sizing devices for selecting annuloplasty rings during surgical repair of cardiac valves.

### Description of the Related Art

Any of the four valves in the heart may become incompetent, causing regurgitant backflow of blood. The incidence of valvular insufficiency is considerable, as 334,000 patients are estimated to have severe aortic insufficiency in the United States (Maumer G. Aortic regurgitation. Heart 2006; 92(7):2480). Valvular insufficiency may be treated by valve replacement or valve repair. Increasing clinical evidence suggests that aortic valve repair may have better long-term outcomes than aortic valve replacement (Meuris B. Starting an aortic valve repair program: is it worthwhile. Aortic valve repair compared to replacement. Eur J Cardiothorac Surg 2021; 60(6):1369).

When valvular insufficiency is the result of annular dilatation, surgical repair involves placement of an annuloplasty ring either internal or external to the heart to reduce the diameter of the annulus and restore valve competency. Selection of the proper size of annuloplasty ring used for valvuloplasty has traditionally been performed during the procedure, using rigid passive sizing devices inserted into the surgically exposed incompetent valve. For aortic valve repair procedures, valve sizers composed of balls of varying diameters are used. For mitral valve repair procedures, discs of varying dimensions matching the shape of the mitral annulus are inserted to obtain the measurement.

Current valve sizing devices measure the dimensions of the valve annulus, which is the insertion site of the valve leaflets. Existing valve annular dimensions provide an approximation for the size of the annuloplasty ring that should be used to reduce the circumference of the annulus to achieve valve leaflet coaptation for correction of valvular regurgitation.

Improved devices and methods for sizing cardiac valve annuli during annuloplasty procedures are desired.

### SUMMARY

Several active annuloplasty sizing devices are described herein, which are useful for selecting annuloplasty rings during surgical repair of cardiac valves utilizing adjustable sizer rings that are temporarily attached to the valve annulus and contracted to achieve valve leaflet coaptation. Methods of sizing a cardiac valve annulus are also described herein, which are used to determine an appropriate annuloplasty ring size based on direct visual observation of valve leaflet apposition during the sizing process. Preferred alternatives are set forth below.

The present disclosure provides an active annuloplasty sizing device, comprising: an outer tube having a proximal end and a distal end; an inner member slidably engaged with the outer tube and capable of extending from the proximal end of the outer tube; and a sizer ring coupled to a distal end of the outer tube, the sizer ring configured to be positioned at a valve annulus and to receive one or more suture ties for temporary attachment to the valve annulus, the sizer ring comprising: an actuator tube; and an actuator extending through the actuator tube and coupled to the inner member; wherein retraction of the inner member relative to the outer tube causes the actuator to decrease a circumference of the sizer ring.

Optionally, the actuator tube further comprises a coil spring inside of its lumen coaxial with the actuator.

Optionally, the coil spring is covered with the actuator tube.

Optionally, the actuator tube comprises a heat shrink material.

Optionally, the coil spring and outer tube are configured such that when a suture tie is secured to the circular form the suture tie is confined between adjacent coils of the coil spring to prevent axial migration of the suture ties along the circular form.

Optionally, the device further comprises a readout disposed along a length of the inner member, the readout configured to indicate a diameter of the circular form.

Optionally, the device further comprises a releasable lock disposed on the actuator handle and configured to engage the inner member to prevent advancement of the inner member while permitting retraction of the inner member.

Optionally, the inner member comprises ratchet teeth configured to interact with the releasable lock.

Optionally, the sizer ring is oriented substantially orthogonal to a longitudinal axis of the outer tube.

Optionally, the actuator comprises a flexible member selected from a suture, a wire, or a polymer filament.

Optionally, the device further comprises one or more handles disposed at the proximal end of the outer tube and at a proximal end of the inner member.

The present disclosure provides a method of sizing a cardiac valve annulus, comprising: placing one or more suture ties at commissures of a cardiac valve; positioning a sizer ring of an active sizing device at the cardiac valve annulus, the active sizing device comprising an outer tube and an inner member slidably engaged with the outer tube; securing the sizer ring to the cardiac valve annulus using the one or more suture ties; retracting the inner member relative to the outer tube to decrease a circumference of the sizer ring and cinch the cardiac valve annulus until valve leaflets of the cardiac valve are observed to be in apposition; and determining an annuloplasty ring size based on a dimension indicated by the active sizing device when the valve leaflets are in apposition.

Optionally, the dimension is indicated by a readout disposed along a length of either the inner member or the outer tube.

Optionally, the sizer ring comprises a coil spring covered with an actuator tube, and the one or more suture ties are confined between adjacent coils of the coil spring.

Optionally, the method further comprises selecting an annuloplasty ring corresponding to the determined annuloplasty ring size.

Optionally, the cardiac valve is an aortic valve and the one or more suture ties are placed at three commissures of the aortic valve.

Optionally, retracting the inner member causes uniform contraction along the circumference of the sizer ring such that relative positions of the commissures are maintained during cinching.

The present disclosure further provides a kit for cardiac annuloplasty sizing, comprising: an active annuloplasty sizing device comprising: an outer tube having a proximal end and a distal end; a sizer ring coupled to the outer tube adjacent the distal end; an inner member slidably engaged with the outer tube and operatively coupled to the sizer ring, wherein retraction of the inner member relative to the actuator handle decreases a circumference of the sizer ring; and a readout disposed along a length of the annuloplasty sizing device to indicate a diameter of the sizer ring; and at least two annuloplasty rings of different diameters.

Optionally, the sizer ring comprises a coil spring covered with an actuator tube, the compression spring and actuator tube configured to confine suture ties between adjacent coils of the compression spring.

Optionally, the active annuloplasty sizing device further comprises a releasable lock disposed on the actuator handle and configured to engage the inner member to prevent advancement of the inner member while permitting retraction of the inner member.

### BRIEF DESCRIPTION OF FIGURES

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIGS. 1a and 1b illustrate front and side views of an active annuloplasty sizing device, according to aspects of the present disclosure.

FIGS. 1c and 1d illustrate side and back views of the active annuloplasty sizing device of FIGS. 1a and 1b, according to aspects of the present disclosure.

FIGS. 2a and 2b illustrate front views of the active annuloplasty sizing device of FIGS. 1a and 1b in two different configurations, according to aspects of the present disclosure.

FIGS. 3a and 3b depict conventional passive annuloplasty sizing devices used during sizing procedures.

FIG. 4a illustrates a sizer ring of the active annuloplasty sizing device being inserted towards an aortic valve, according to aspects of the present disclosure.

FIG. 4b illustrates the sizer ring fixed in an expanded configuration at an aortic annulus, according to aspects of the present disclosure.

FIG. 4c depicts retraction of an inner member relative to an outer tube during a sizing process, according to aspects of the present disclosure.

FIGS. 5a and 5b illustrate cross-sectional views of internal and external annuloplasty ring placements, according to aspects of the present disclosure.

FIG. 6 illustrates a front view of another active annuloplasty sizing device, according to aspects of the present disclosure.

### DETAILED DESCRIPTION

A description of example embodiments follows. Like numbers refer to like elements throughout.

Embodiments will now be described with reference to the accompanying drawings, which show some, but not all of the disclosed embodiments. While this invention has been particularly shown and described with references to example embodiments thereof, it will be understood by those skilled in the art that various changes in form and details may be made therein without departing from the scope of the invention encompassed by the appended claims. Furthermore, while several embodiments are described, the scope of the embodiments should not be construed to be limited to those set forth herein.

For the purposes of this disclosure, the terms “proximal” and “distal” are used with reference to the device, that is, “proximal” denotes the region near the handles of the device where the surgeon manipulates the inner member and outer tube, and “distal” denotes the region away from the handles toward and beyond the sizer ring that is positioned at the valve annulus.

Cardiac valves may become incompetent due to various conditions, including annular dilatation, which causes regurgitant backflow of blood through the valve. Surgical repair of incompetent cardiac valves may involve placement of an annuloplasty ring to reduce the diameter of the valve annulus and restore valve competency. Annuloplasty rings may be positioned either internal or external to the heart. Selection of an appropriate annuloplasty ring size during surgical valve repair has traditionally been performed using passive sizing devices that measure existing valve annular dimensions to approximate the annuloplasty ring size.

The present disclosure describes active annuloplasty sizing devices and methods that allow a surgeon to determine an appropriate annuloplasty ring size based on direct visual observation of valve leaflet apposition during the sizing process. An active annuloplasty sizing device may comprise an outer tube having a proximal end and a distal end, and an inner member slidably engaged with the outer tube and capable of extending from the proximal end of the outer tube. A sizer ring may be coupled to a distal end of the outer tube. The sizer ring may be configured to be positioned at a valve annulus and to receive one or more suture ties for temporary attachment to the valve annulus. The sizer ring may comprise an actuator tube and an actuator extending through the actuator tube and coupled to the inner member. Retraction of the inner member relative to the outer tube may cause the actuator to decrease a circumference of the sizer ring.

A method of sizing a cardiac valve annulus may comprise placing one or more suture ties at commissures of a cardiac valve, and positioning a sizer ring of an active sizing device at the cardiac valve annulus. The active sizing device may comprise an outer tube and an inner member slidably engaged with the outer tube. The method may further comprise securing the sizer ring to the cardiac valve annulus using the one or more suture ties, and retracting the inner member relative to the outer tube to decrease a circumference of the sizer ring and cinch the cardiac valve annulus until valve leaflets of the cardiac valve are observed to be in apposition. An annuloplasty ring size may be determined based on a dimension indicated by the active sizing device when the valve leaflets are in apposition.

A kit for cardiac annuloplasty sizing may comprise an active annuloplasty sizing device and multiple annuloplasty rings of different diameters. The active annuloplasty sizing device of the kit may comprise an outer tube having a proximal end and a distal end, a sizer ring coupled to the outer tube adjacent the distal end, and an inner member slidably engaged with the outer tube and operatively coupled to the sizer ring. Retraction of the inner member relative to the actuator handle may decrease the circumference of the sizer ring. A readout may be disposed along a length of the annuloplasty sizing device to indicate an appropriate diameter of the sizer ring.

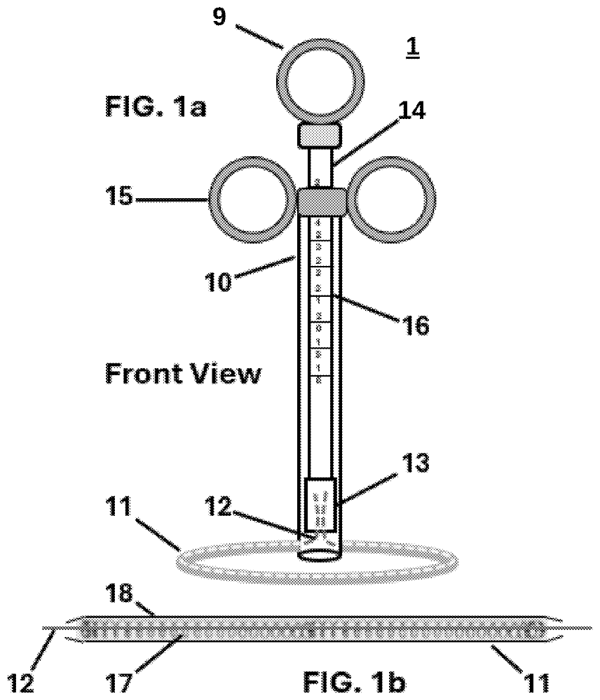

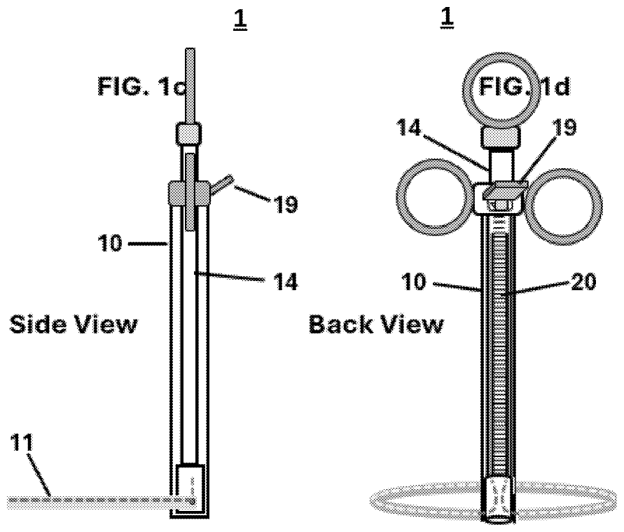

FIGS. 1a and 1b illustrate an active annuloplasty sizing device 1. The front view shown in FIG. 1a depicts an outer tube 10 with an attached sizer ring 11 mounted near a distal end of the outer tube 10. The outer tube 10 may be any suitable elongate member having a lumen, and a cross-sectional shape of the outer tube 10 may be any suitable shape such as rectangular or circular, for example. The sizer ring 11 is a flexible, substantially circular ring that is orthogonally situated near the distal end of the outer tube 10. In some embodiments, the sizer ring 11 may be oriented substantially orthogonal to a longitudinal axis of the outer tube 10.

An actuator 12 extends through a lumen of the sizer ring 11 and is coupled to an inner member 14 via a bushing 13 on a distal end of the inner member 14. However, in some embodiments, the actuator 12 may be attached directly to the inner member 14. The actuator 12 is a flexible tensile member and may be a suture, a wire, or a polymer filament. In some embodiments, the actuator 12 comprises a monofilament polymer suture constructed of nylon with a cross-sectional diameter of approximately 1.0 mm. In some embodiments, the actuator 12 comprises a monofilament polymer suture constructed of polypropylene. In this embodiment, the actuator 12 attaches to the inner member 14 via the bushing 13 on the distal end of the inner member 14 and extends through an entire length of a lumen of a coil spring 17 within the sizer ring 11.

The inner member 14 is slidably engaged with the outer tube 10 may extend a full length of or out of a proximal end of the outer tube 10 or in some embodiments it may terminate before the distal end of the outer tube 10. An outer tube handle 15 may be attached to the proximal end of the outer tube 10, and an inner member handle 9 may be attached to the proximal end of the inner member 14 to facilitate advancement and retraction of the inner member 14 relative to the outer tube 10. The outer tube handle 15 and the inner member handle 9 may comprise finger rings attached to the proximal ends to facilitate advancement and retraction of the inner member 14 relative to the outer tube 10. While finger rings are shown, in some embodiments, other suitable grasping features may be used in place of handles, such as levers, beams, or grips.

The inner member 14 contains a readout 16 along a length of the inner member 14. The readout 16 is configured to indicate a diameter of the sizer ring 11 to a surgeon during an active sizing process. In some embodiments, the readout 16 may contain any features that indicate valve sizing such as numbers (i.e., diameter), letters, words, or markings that indicate which valve implant should be chosen. In embodiments disclosed herein, the readout may have lines or labels on either or both of the slidably engaged members-inner member and outer tube. For example, the outer tube may have one or more marks that line up with marks and labels on the inner member to indicate a size. Conversely, the inner member may have one or more marks that line up with marks and labels on the outer tube to indicate a size.

FIG. 1b depicts a section of the sizer ring 11 in greater detail. The sizer ring 11 comprises an actuator tube 18 and the coil spring 17 inside of a lumen of the actuator tube 18 coaxial with the actuator 12. The coil spring 17 is covered with the actuator tube 18. In some embodiments, the coil spring 17 of the sizer ring 11 is a stainless steel compression spring with an outer diameter of approximately 3 mm. In some embodiments, the coil spring 17 of the sizer ring 11 has a wire diameter of approximately 0.5 mm. The coil spring 17 may provide an outward bias to the sizer ring 11.

The actuator tube 18 may comprise a heat shrink material. In some embodiments, the actuator tube 18 comprises a polyethylene terephthalate (PET) heat shrink tube with a wall thickness of approximately 0.013 mm. The actuator 12 resides in the lumen of the coil spring 17.

The actuator tube 18 covered coil spring 17 configuration of the sizer ring 11 prevents or reduces migration of commissural suture ties along the sizer ring 11 during a cinching process. The coil spring 17 and outer tube 10 are configured such that when a suture tie is secured to the sizer ring 11, the suture tie is confined between adjacent coils of the coil spring 17 to prevent axial migration of the suture ties along the sizer ring 11, e.g., while the sizer ring is articulated by the actuator 12. Each suture strand tied onto the actuator tube 18 ensheathed coil spring 17 is confined by adjacent spiral coils that prevent axial movement along the coil spring 17. As suture strands are tied onto the sizer ring 11, recesses in between the coils of the coil spring 17 retain the suture strands such that the suture strands are captive between the coils.

FIGS. 1c and 1d illustrate side and back views, respectively, of the active annuloplasty sizing device 1. The side view shown in FIG. 1c depicts the outer tube 10 with the inner member 14 extending through a length of the outer tube 10. A ratchet lock 19 is positioned near the proximal end of the outer tube 10. The sizer ring 11 is shown orthogonally attached near the distal end of the outer tube 10.

The back view shown in FIG. 1d provides additional detail of the device configuration. The inner member 14 extends through the outer tube 10 and features ratchet teeth 20 along a back surface of the inner member 14. The ratchet teeth 20 are configured to interact with the ratchet lock 19 on the proximal end of the outer tube 10. The ratchet teeth 20 are angled in a direction that permits unimpeded retraction of the inner member 14 while preventing advancement of the inner member 14 unless the ratchet lock 19 is depressed. The ratchet lock 19 may function as a releasable lock disposed on the outer tube 10 and configured to engage the inner member 14 to prevent advancement of the inner member 14 while permitting retraction of the inner member 14. The ratchet mechanism allows the inner member 14 to be retracted to decrease the diameter of the sizer ring 11 while locking the inner member 14 against extension to increase the diameter of the sizer ring 11 until the ratchet lock 19 is released. Hence, the ratchet lock 19 maintains the diameter of the sizer ring 11 as the diameter is incrementally decreased during active cinching of the valve annulus during the sizing process.

A ratchet lock mechanism is one way of achieving a releasable lock on the inner member 14. Other locking features may be used, such as friction-based locks having a spring that biases a locking element, such as a cam or plunger, against the inner member 14.

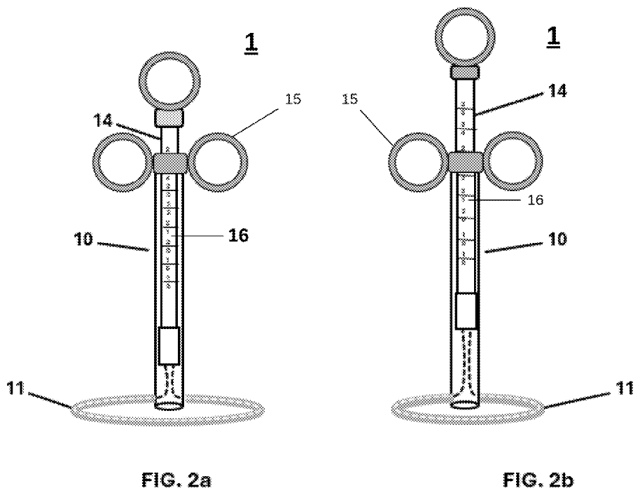

FIGS. 2a and 2b illustrate front views of the active annuloplasty sizing device 1 in two different configurations, demonstrating an actuation mechanism for adjusting a diameter of the sizer ring 11. In FIG. 2a, the active annuloplasty sizing device 1 is shown in an initial configuration with the inner member 14 fully advanced into the outer tube 10. In this state, the sizer ring 11 is positioned at a maximal diameter. In some embodiments, the sizer ring 11 may be oriented substantially orthogonal to a longitudinal axis of the outer tube 10 as shown. The outer tube 10 includes an outer tube handle 15 positioned at a proximal end of the outer tube 10 to facilitate manipulation of the active annuloplasty sizing device 1. The inner member 14 extends from the proximal end of the outer tube 10 and includes a readout 16 along a length of the inner member 14 that indicates, for example, the diameter of the sizer ring 11 during a sizing process.

In FIG. 2b, the active annuloplasty sizing device 1 is shown with the inner member 14 retracted out of the outer tube 10, resulting in a decreased diameter of the sizer ring 11 compared to the configuration shown in FIG. 2a. Retraction of the inner member 14 relative to the outer tube 10 causes uniform contraction along a circumference of the sizer ring 11. In some embodiments, retracting the inner member 14 causes uniform contraction along the circumference of the sizer ring 11 such that relative positions of commissures are maintained during cinching. The readout 16 on the inner member 14 provides a surgeon with a visual indication of a corresponding annular dimension as the sizer ring 11 is contracted during an active sizing procedure.

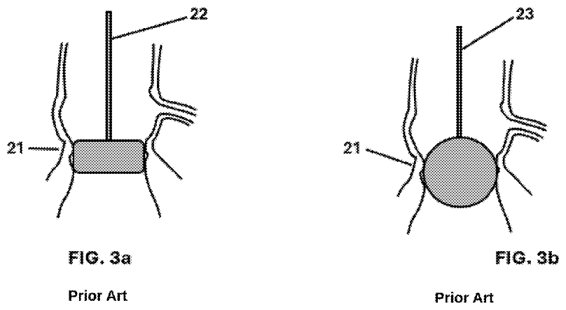

FIGS. 3a and 3b depict conventional passive annuloplasty sizing devices used during sizing procedures. FIG. 3a shows a conventional passive disc annuloplasty sizing device 22 inserted to the level of an aortic annulus 21 during a sizing process. The conventional passive disc annuloplasty sizing device 22 comprises an elongated handle with a disc-shaped element at a distal end of the elongated handle that is positioned within the aortic annulus 21 to measure annular dimensions. FIG. 3b depicts a passive ball annuloplasty device 23 introduced into the aortic annulus 21 for sizing during an annuloplasty procedure. The passive ball annuloplasty device 23 includes an elongated handle with a spherical element at a distal end of the elongated handle that is positioned within the aortic annulus 21. The spherical configuration of the passive ball annuloplasty device 23 allows measurement of the valve annulus by fitting the spherical element into an annular opening. Both the conventional passive disc annuloplasty sizing device 22 and the passive ball annuloplasty device 23 represent rigid passive sizing devices that measure existing valve annular dimensions to provide an approximation for selecting an appropriate annuloplasty ring size.

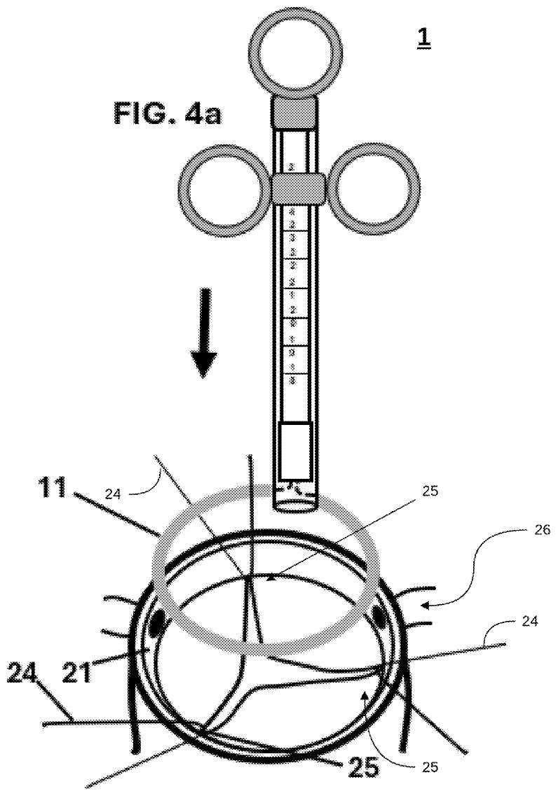

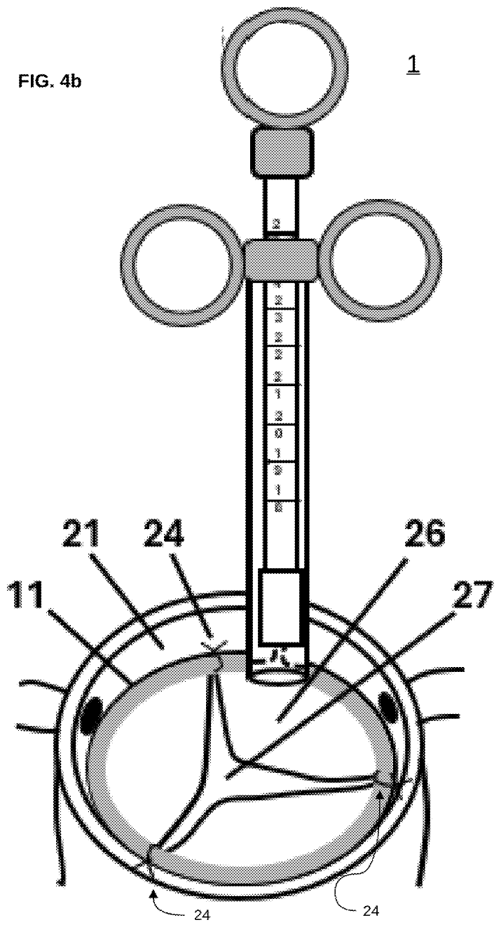

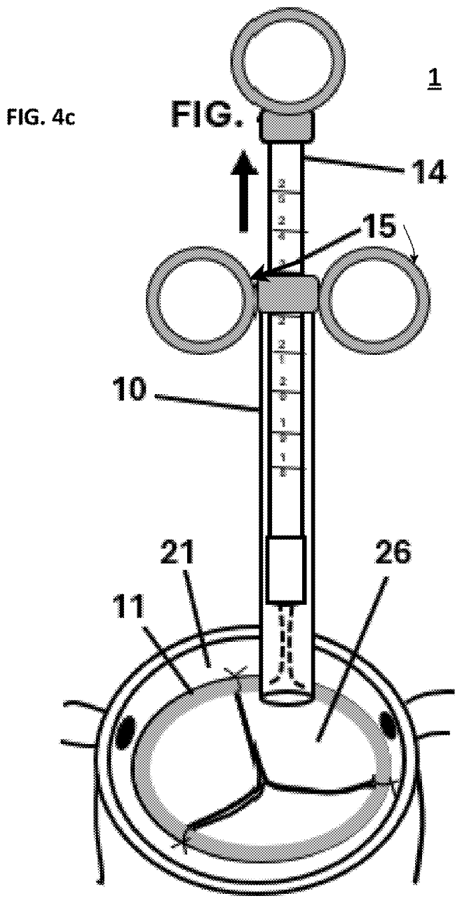

FIG. 4a illustrates a sizer ring 11 of the active annuloplasty sizing device 1 being inserted toward a level of an aortic annulus 21 of a surgically exposed aortic valve 26. Prior to insertion of the sizer ring 11, commissural suture ties 24 are placed at commissures 25 of the aortic valve 26. The commissures 25 are points at which adjacent valve leaflets insert into the valve annulus. In a tricuspid aortic valve configuration, three commissures 25 are present. A suture strand armed with a tapered point curved needle may be used to place a suture at each commissure 25. The sizer ring 11 is encircled by the commissural suture ties 24 to secure the active annuloplasty sizing device 1 at an annular level during an active sizing process. As described above, the inner member 14 includes a readout 16 along a length of the inner member 14 that corresponds to a diameter of the sizer ring 11, allowing a surgeon to determine an appropriate annuloplasty ring size during the procedure.

FIG. 4b illustrates the sizer ring 11 of the active annuloplasty sizing device 1 fixed in an expanded configuration at the aortic annulus 21. The sizer ring 11 is secured in position by the commissural suture ties 24, which have been placed at the commissures of the aortic valve. In this configuration, valve leaflets 26 are not in apposition with one another, resulting in an incompetent valve condition. The lack of proper leaflet coaptation creates a sizeable opening 27 between the valve leaflets 26, which represents a regurgitant gap that allows backflow of blood through the incompetent valve. FIG. 4b depicts the state of the valve prior to cinching of the sizer ring 11, demonstrating the condition that may be addressed by annular reduction to achieve proper valve leaflet apposition and restore valvular competency.

FIG. 4c depicts retraction of the inner member 14 relative to the outer tube 10 of the active annuloplasty sizing device 1 during the sizing process. The outer tube 10 extends vertically with an outer tube handle 15 positioned at the proximal end to facilitate manipulation by a surgeon. The inner member 14 is shown being retracted in an upward direction, with a readout 16 visible along a length of the inner member 14 to indicate, for example, a diameter of the sizer ring 11 during the active sizing process. The sizer ring 11 is positioned at the aortic annulus 21 of the surgically exposed aortic valve.

Retraction of the inner member 14 relative to the outer tube 10 causes the sizer ring 11 to cinch down the aortic annulus 21 until valve leaflets 26 come into apposition to correct the leakage. As described above, the sizer ring 11 may comprise a coil spring 17 covered with an actuator tube 18, and the one or more commissural suture ties 24 may be confined between adjacent coils of the coil spring 17 to prevent axial migration along the sizer ring 11 during the cinching process. The valve leaflets 26 are shown in apposition in FIG. 4c, demonstrating correction of the previously incompetent valve. At this configuration, the dimension indicated by the readout 16 on the inner member 14 corresponds to the annuloplasty ring size that results in valve leaflet apposition and valvular competency observed directly by the surgeon during the procedure. The dimension may be indicated by the readout 16 disposed along a length of either the inner member 14 or the outer tube 10. Based on the dimension indicated by the readout 16 when the valve leaflets 26 are in apposition, a surgeon may select an annuloplasty ring corresponding to the determined annuloplasty ring size for performance of the annuloplasty procedure.

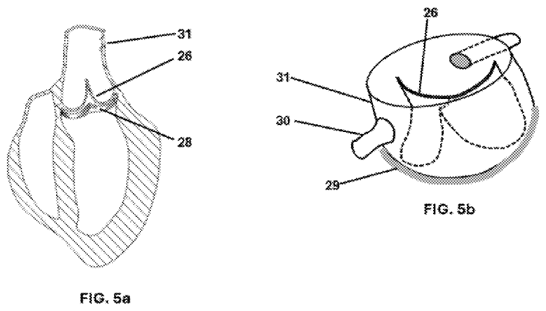

FIGS. 5a and 5b illustrate cross-sectional views of internal and external annuloplasty ring placements relative to valve leaflets 26. The active annuloplasty sizing devices described herein may be used for mitral valve repair procedures in addition to aortic valve repair procedures.

FIG. 5a depicts an internal annuloplasty ring 28 positioned in a sub-valvular location relative to the valve leaflets 26. The internal annuloplasty ring 28 is seated within an annular region beneath the valve leaflets 26. A root of the aorta 31 extends upward from the valve structure. The internal annuloplasty ring 28 may be placed inside the valve in a sub-valvular position with respect to the valve leaflets 26.

FIG. 5b depicts an external annuloplasty ring 29 and a spatial relationship of the external annuloplasty ring 29 to the valve leaflets 26. The external annuloplasty ring 29 is positioned around an exterior of an aortic root structure. A trunk of a coronary artery 30 is visible emanating from the root of the aorta 31. The external annuloplasty ring 29 encircles an outside of the heart to provide support for the valve annulus. The external annuloplasty ring 29 may be placed outside the heart as an alternative to internal placement as shown in FIG. 5a.

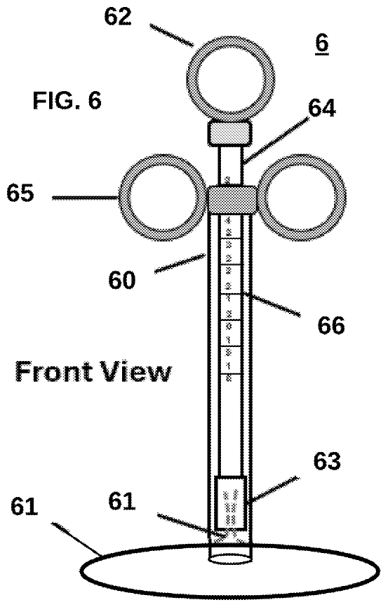

FIG. 6 illustrates a front view of an active annuloplasty sizing device 6. The active annuloplasty sizing device 6 includes an outer tube 60 forming a main body of the active annuloplasty sizing device 6. An inner member 64 is slidably engaged with the outer tube 60 and extends from a proximal end of the active annuloplasty sizing device 6. An inner tube handle 62 is attached to a proximal end of the inner member 64, and an outer tube handle 65 is attached to a proximal end of the outer tube 60 to facilitate advancement and retraction of the inner member 64 relative to the outer tube 60. A readout 66 is disposed along a length of the inner member 64 and is configured to indicate a diameter of an actuator 61 during an active sizing process. A bushing 63 is positioned at a distal end of the inner member 64.

The actuator 61 is coupled to a distal end of the inner member 64 and is oriented substantially orthogonal to a longitudinal axis of the active annuloplasty sizing device 6. The actuator 61 is configured to be positioned at a valve annulus and to receive suture ties for temporary attachment to the valve annulus. Retraction of the inner member 64 relative to the outer tube 60 causes the actuator 61 to decrease in circumference, thereby cinching the valve annulus during an active sizing procedure. The actuator 61 may be made of a wire that may be pushed and pulled to various diameters. Candidate materials for the actuator 61 include metals such as stainless steel or nitinol, or polymers. The actuator 61 may have a predefined shape that is biased like a hoop such that when the actuator 61 is fed out of the outer tube 60, the actuator 61 approximates a circular shape. In some embodiments, a shape-memory or superelastic material may be used for the actuator 61.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended alternatives and equivalents thereto, inventive embodiments may be practiced otherwise than as specifically described. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. An active annuloplasty sizing device, comprising:
an outer tube having a proximal end and a distal end;
an inner member slidably engaged with the outer tube and capable of extending from the proximal end of the outer tube; and
a sizer ring coupled to a distal end of the outer tube, the sizer ring configured to be positioned at a valve annulus and to receive one or more suture ties for temporary attachment to the valve annulus, the sizer ring comprising:
an actuator tube; and
an actuator extending through the actuator tube and coupled to the inner member;
wherein retraction of the inner member relative to the outer tube causes the actuator to decrease a circumference of the sizer ring.

2. The device of claim 1, wherein the actuator tube further comprises a coil spring inside of the its lumen coaxial with the actuator.

3. The device of claim 2, wherein the coil spring is covered with the actuator tube.

4. The device of claim 3, wherein the actuator tube comprises a heat shrink material.

5. The device of claim 3, wherein the coil spring and outer tube are configured such that when a suture tie is secured to the circular form the suture tie is confined between adjacent coils of the coil spring to prevent axial migration of the suture ties along the circular form.

6. The device of claim 1, further comprising a readout disposed along a length of the inner member, the readout configured to indicate a diameter of the circular form.

7. The device of claim 1, further comprising a releasable lock disposed on the actuator handle and configured to engage the inner member to prevent advancement of the inner member while permitting retraction of the inner member.

8. The device of claim 7, wherein the inner member comprises ratchet teeth configured to interact with the releasable lock.

9. The device of claim 1, wherein the sizer ring is oriented substantially orthogonal to a longitudinal axis of the outer tube.

10. The device of claim 1, wherein the actuator comprises a flexible member selected from a suture, a wire, or a polymer filament.

11. The device of claim 1, further comprising one or more handles disposed at the proximal end of the outer tube and at a proximal end of the inner member.

12. A method of sizing a cardiac valve annulus, comprising:
placing one or more suture ties at commissures of a cardiac valve;
positioning a sizer ring of an active sizing device at the cardiac valve annulus, the active sizing device comprising an outer tube and an inner member slidably engaged with the outer tube;
securing the sizer ring to the cardiac valve annulus using the one or more suture ties;
retracting the inner member relative to the outer tube to decrease a circumference of the sizer ring and cinch the cardiac valve annulus until valve leaflets of the cardiac valve are observed to be in apposition; and
determining an annuloplasty ring size based on a dimension indicated by the active sizing device when the valve leaflets are in apposition.

13. The method of claim 12, wherein the dimension is indicated by a readout disposed along a length of either the inner member or the outer tube.

14. The method of claim 12, wherein the sizer ring comprises a coil spring covered with an actuator tube, and wherein the one or more suture ties are confined between adjacent coils of the coil spring.

15. The method of claim 12, further comprising selecting an annuloplasty ring corresponding to the determined annuloplasty ring size.

16. The method of claim 12, wherein the cardiac valve is an aortic valve and the one or more suture ties are placed at three commissures of the aortic valve.

17. The method of claim 12, wherein retracting the inner member causes uniform contraction along the circumference of the sizer ring such that relative positions of the commissures are maintained during cinching.

18. A kit for cardiac annuloplasty sizing, comprising:
an active annuloplasty sizing device comprising:
an outer tube having a proximal end and a distal end;
a sizer ring coupled to the outer tube adjacent the distal end;
an inner member slidably engaged with the outer tube and operatively coupled to the sizer ring, wherein retraction of the inner member relative to the actuator handle decreases a circumference of the sizer ring; and
a readout disposed along a length of the annuloplasty sizing device to indicate a diameter of the sizer ring; and
at least two annuloplasty rings of different diameters.

19. The kit of claim 18, wherein the sizer ring comprises a coil spring covered with an actuator tube, the compression spring and actuator tube configured to confine suture ties between adjacent coils of the compression spring.

20. The kit of claim 18, wherein the active annuloplasty sizing device further comprises a releasable lock disposed on the actuator handle and configured to engage the inner member to prevent advancement of the inner member while permitting retraction of the inner member.
